(19) United States
(12) Patent Application Publication
(10) Pub. No.: US 2007/0118099 A1
(43) Pub. Date: May 24, 2007

(54) Method and apparatus for endovascular graft cutting

(76) Inventor: Hugh H. Trout III, Bethesda, MD (US)

Correspondence Address: KELLEY DRYE & WARREN LLP, 3050 K STREET, NW, SUITE 400, WASHINGTON, DC, 20007, US

(21) Appl. No.: 11/503,922

(22) Filed: Aug. 15, 2006

Related U.S. Application Data
(60) Provisional application No. 60/707,943, filed on Aug. 15, 2005.

(51) Int. Cl. A61B 18/04; A61B 18/14

(52) U.S. Cl. 606/27; 606/45

(57) ABSTRACT

The present invention is directed to a method and apparatus for cutting endografts endovascularly. An embodiment of the present invention is to develop a method and apparatus to cut an unsupported endograft after the endograft has been inserted into the artery for the repair of an abdominal aortic aneurysm.

### CROSS-REFERENCE TO RELATED APPLICATIONS

The present invention relates to, and is entitled to the benefit of the earlier filing date and priority of U.S. Application No. 60/707,943 filed Aug. 15, 2005.

### FIELD OF THE INVENTION

The present invention relates generally an apparatus and method for use in surgical repair, more particularly for endovascular cutting of surgical grafts.

### BACKGROUND

An aneurysm is a ballooning of the wall of an artery resulting from the weakening of the artery due to disease or other conditions. Left untreated, the aneurysm will frequently rupture, resulting in loss of blood through the rupture and death.

Aortic aneurysms are the most common form of arterial aneurysm and are life threatening. The aorta is the main artery which supplies blood to the circulatory system. The aorta arises from the left ventricle of the heart, passes upward and bends over behind the heart, and passes down through the thorax and abdomen. Among other arterial vessels branching off the aorta along its path, the abdominal aorta supplies two side vessels to the kidneys, the renal arteries. Below the level of the renal arteries, the abdominal aorta continues to about the level of the fourth lumbar vertebrae (or the navel), where it divides into the iliac arteries. The iliac arteries, in turn, supply blood to the lower extremities and perineal region.

It is common for an aortic aneurysm to occur in that portion of the abdominal aorta between the renal arteries and the iliac arteries. This portion of the abdominal aorta is particularly susceptible to weakening, resulting in an aortic aneurysm. Such an aneurysm is often located near the iliac arteries. An aortic aneurysm larger than about 5 cm in diameter in this section of the aorta is ominous. Left untreated, the aneurysm may rupture, resulting in rapid, and usually fatal, hemorrhaging. Typically, a surgical procedure is not performed on aneurysms smaller than 5 cm as no statistical benefit exists to do so.

Aneurysms in the abdominal aorta are associated with a particularly high mortality rate; accordingly, current medical standards call for urgent operative repair. Abdominal surgery, however, results in substantial stress to the body. Although the mortality rate for an aortic aneurysm is extremely high, there is also considerable mortality and morbidity associated with open surgical intervention to repair an aortic aneurysm. This intervention involves penetrating the abdominal wall to the location of the aneurysm to reinforce or replace the diseased section of the abdominal wall (i.e., abdominal aorta). A prosthetic device, typically a synthetic tube graft, is used for this purpose. The graft serves to exclude the aneurysm from the circulatory system, thus relieving pressure and stress on the weakened section of the aorta at the aneurysm.

Repair of an aortic aneurysm by surgical means is a major operative procedure. Substantial morbidity accompanies the procedure, resulting in a protracted recovery period. Further, the procedure entails a substantial risk of mortality. While surgical intervention may be indicated and the surgery carries attendant risk, certain patients may not be able to tolerate the stress of intra-abdominal surgery. It is, therefore, desirable to reduce the mortality and morbidity associated with intra-abdominal surgical intervention.

In recent years, methods have been developed to attempt to treat an abdominal aortic aneurysm without the attendant risks of intra-abdominal surgical intervention. Although techniques have been developed that may reduce the stress, morbidity, and risk of mortality associated with surgical intervention to repair aortic aneurysms, none of the prior art systems that have been developed effectively treat the aneurysm and exclude the affected section of aorta from the pressures and stresses associated with circulation. None of the devices disclosed in the references provide a reliable and quick means to reinforce an aneurysmal artery. In addition, all of the prior references require a sufficiently large section of healthy aorta abutting the aneurysm to ensure attachment of the graft. The proximal aortic neck (i.e., above the aneurysm) is usually sufficient to support a graft's attachment means. However, when an aneurysm is located near the iliac arteries, there may be an ill-defined neck or no neck below the aneurysm. Such an ill-defined neck would have an insufficient amount of healthy aortic tissue to which to successfully attach a graft. Furthermore, much of the abdominal aortic wall may be calcified making it extremely difficult to attach a graft thereto.

Additionally, there are occasions when it is advantageous to use an unsupported endograft. A new approach to the endovascular treatment of aortic aneurysms involves using only unsupported endografts where the unsupported endograft can be inserted and the tube portion attached to the aortic neck and the distal limbs attached to the iliac arteries with commercially available stents. An endovascular approach using unsupported endografts would substantially lower costs associated with the procedure because a current supported endograft typically costs about $20,000. A problem with this approach is that the unsupported endograft must be cut before it is inserted into the body because there is no currently available method to cut an unsupported endograft endovascularly. Because it is impossible to know the exact length needed for the limbs of the endograft without completing some type of preoperative imaging study, there is a need to develop a method and apparatus to cut the endografts after they have been inserted into the artery. There is a need in the industry to develop an apparatus and method to trim excess graft material from an endograft following placement of the endograft at the surgical site.

Additional advantages of various embodiments of the invention are set forth, in part, in the description that follows and, in part, will be apparent to those of ordinary skill in the art from the description and/or from the practice of the invention.

### SUMMARY

Embodiments of the present invention are directed to a method and apparatus for cutting endografts endovascularly. One embodiment of the present invention is to develop a method and apparatus to cut an unsupported endograft after the endograft has been inserted into the artery for the repair of an aortic aneurysm, including, but not limited to, an abdominal aortic aneurysm.

Further embodiments of the method and apparatus of using the present invention include using the stent that is inserted into the distal limbs of the unsupported endograft to cut the unsupported endograft. In one embodiment, a current is applied to a filament imbedded in the outside portion of the distal end of the stent that will heat the filament sufficiently to burn through the material of the endograft that is in the immediate contact with the distal end of the stent.

One embodiment of an apparatus for endovascularly cutting a graft comprises a catheter having a first end, a second end, an inner lumen, and an outer surface, further comprising at least one opening near its first end, at least one wire further comprising a filament extending through the at least one opening and around an outer surface of the catheter, wherein the wire is movable within the catheter and can be extended to form a ring disposed a predetermined distance around the outer surface of the catheter.

One embodiment of an apparatus for endovascularly cutting a graft comprises a stent having a distal end, a catheter having a first end, a second end, an inner lumen, and an outer surface, a filament disposed within the circumference of the distal end of the stent, and a wire having a first end and a second end, wherein the first end is in communication with the filament and the second end extends away from the stent into the lumen of the catheter.

One embodiment of an apparatus for endovascularly cutting a graft comprises a flared sheath having a first flared end, a second end, an inner lumen, and an outer surface, a catheter having a first end, a second end, an inner lumen, and an outer surface, wherein a portion of the catheter is disposed within the inner lumen of the flared sheath, an inner sheath having a first end, a second end, an inner lumen, and an outer surface, wherein a portion of the inner sheath is disposed within the inner lumen of the catheter, and an optical fiber having a first end and a second end, wherein a portion of the optical fiber is disposed within the inner sheath.

One embodiment of of the present invention is a method for endovascularly cutting a graft comprising the steps of inserting a catheter having a first end, a second end, an inner lumen, and an outer surface, further comprising at least one opening near its first end and at least one wire further comprising a filament extending through the at least one opening and around an outer surface of the catheter, extending the wire comprising the filament to form a ring disposed a predetermined distance around the outer surface of the catheter and contacting the filament to a portion of the graft to be cut, and applying a current to the wire and the filament such that the filament cuts the graft.

An embodiment of the method and apparatus of the present invention includes using a catheter to cut the unsupported endograft. In one embodiment, the catheter includes an optical fiber that is circumferentially rotated to cut the unsupported endograft near the distal end of the inserted stent. In an additional embodiment, the catheter contains a ring with a heated filament that is expanded radially to cut the unsupported endograft.

Additional advantages of the invention will be set forth in part in the description that follows, and in part will be obvious from the description, or may be learned by practice of the invention. The advantages of the invention will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments of the invention and together with the description, serve to explain the principles of the invention. Where appropriate, the same reference numerals refer to the same or similar elements.

FIG. 1 is a schematic view of a supported endograft for an abdominal aortic aneurysm.

FIG. 2-4 are schematic views of an unsupported endograft for an abdominal aortic aneurysm held by a rigid guidewire.

FIG. 5-6 are schematic views of an unsupported endograft for an abdominal aortic aneurysm showing the top of the endograft attached to the aorta neck wall.

FIGS. 7-8 are schematic views showing the insertion of a stent into an iliac artery.

FIG. 9 is a schematic view of a stent expanded in an iliac artery.

FIG. 10 is a schematic view showing the transection of the unsupported endograft limb by the distal end of the stent.

FIG. 11 is schematic view of the stent and the endograft limb after the transection.

FIGS. 12-14 are schematic views of the transection of the endograft limb using a catheter.

FIGS. 15-17 are schematic views of an alternate embodiment for the transection of the endograft limb using a catheter.

FIG. 18 is a schematic view of a catheter of an embodiment of the present invention.

FIG. 19 is a top view of a catheter of an embodiment of the present invention.

### DETAILED DESCRIPTION OF EMBODIMENTS OF THE INVENTION

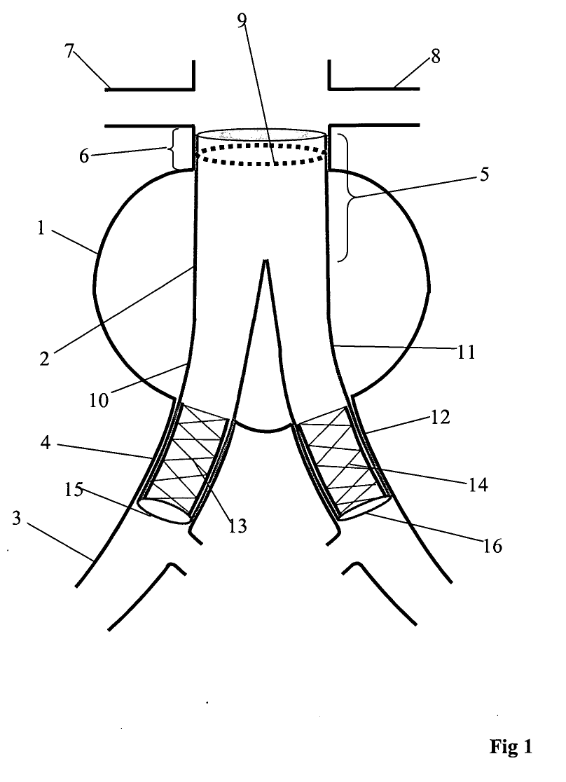

FIG. 1 shows that one method to treat a patient with an aortic aneurysm 1, for example, an abdominal aortic aneurysm (AAA), is to insert prosthetic bifurcation endograft 2, sometimes also referred to, but not limited to, an “endograft”, through external iliac artery 3 and into right common iliac artery 4 and/or left common iliac artery 12 and attach proximal tube portion 5 of endograft 2 to the undilated portion of aorta 6, also referred to as the “aortic neck”, below right renal artery 7 and left renal artery 8. One method to attach the top of tube portion 5 of endograft 2 is to place surgical fasteners 9 as described in U.S. Pat. Nos. 5,957,940; 5,997,556; 6,248,118; 6,520,974; and 6,635,066 and U.S. Patent Application Nos. 60/537,888 and 60/538,242, herein incorporated in their entirety by reference. Endograft 2 may also be attached to aorta 6 by sutures, fasteners, staples, hooks, or any other suitable attachment method. Right bifurcation limb 10 and left bifurcation limb 11 be attached distally to right common iliac artery 4 and left common iliac artery 12 with stents 13 and 14, respectively. These limbs can be cut to their proper length, based on measurements from an imaging study such as computed tomography (CT scan), prior to endograft insertion. An embodiment of the present invention is an apparatus and method to allow the interventionalist inserting the endograft to insert a endograft whose limbs may be too long for the anatomy of the patient and then, after endograft 2 is inserted and attached to aortic neck 6, be able to place stents 13 and 14 and then transect, cut, and/or trim right 10 and left 11 endograft limbs at distal end 15 and 16 of stents 13 and 14, respectively. In this manner endograft 2 can be better matched to the anatomy found during actual endograft insertion without the need for precise preoperative measurements of the endograft limbs. This allows the interventionalist to customize the graft to the individual patient following the placement of the endograft at the surgical site.

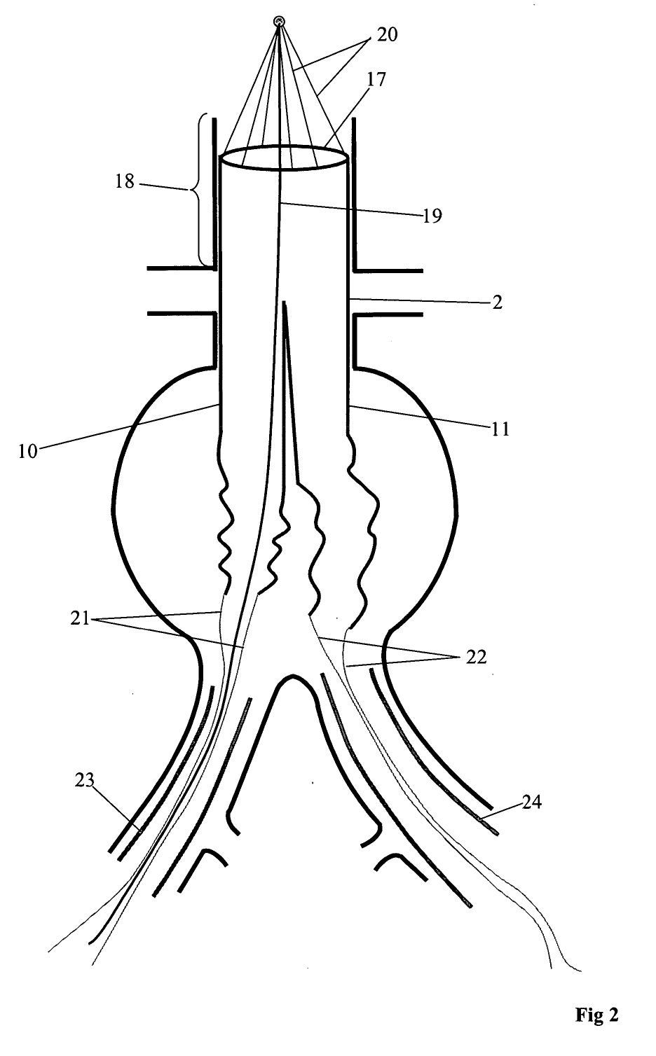

FIG. 2 shows top 17 of endograft 2 being held in suprarenal aorta 18 by guidewire 19 attached to struts 20 which, in turn, are attached to top 17 of endograft 2. Right 10 and left 11 limbs of endograft 2 are within aneurysm I and are attached to sutures 21 and 22 that pass through right 23 and left 24 insertion sheaths that have previously been inserted through the right and left femoral arteries (not shown).

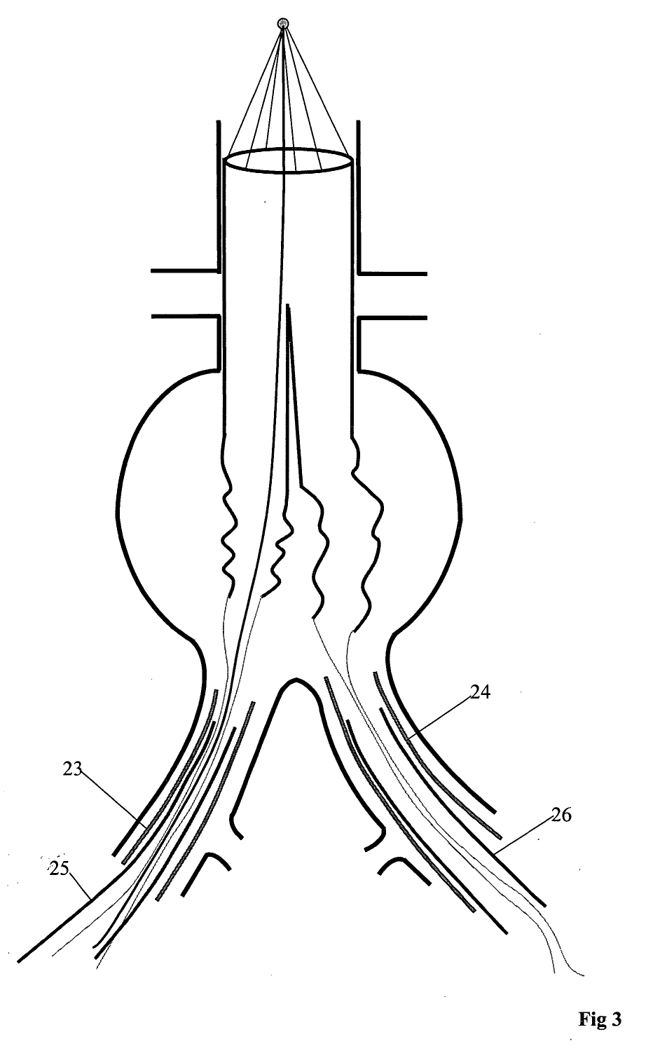

FIG. 3 shows right 25 and left 26 flared sheaths that have been inserted through insertion sheaths 23 and 24 respectively. Flared sheaths 25 and 26 have a first end, second end, inner lumen and an outer surface. The first end of flared sheaths 25 and 26 are inserted into insertion sheaths 23 and 24 and are designed or biased to flair outward when unconstrained. In an embodiment they are designed to be heat resistant such that they will protect tissue contacting their outer diameter even when a hot filament is compressed against their inner diameter. Flared sheaths 25 and 26 may be composed of a metal, such as, but not limited to, stainless steel, and/or Nitinol, and/or any number of well known plastic or polymer materials, such as, but not limited to Teflon or any number of polyamide materials with the necessary heat resistant properties.

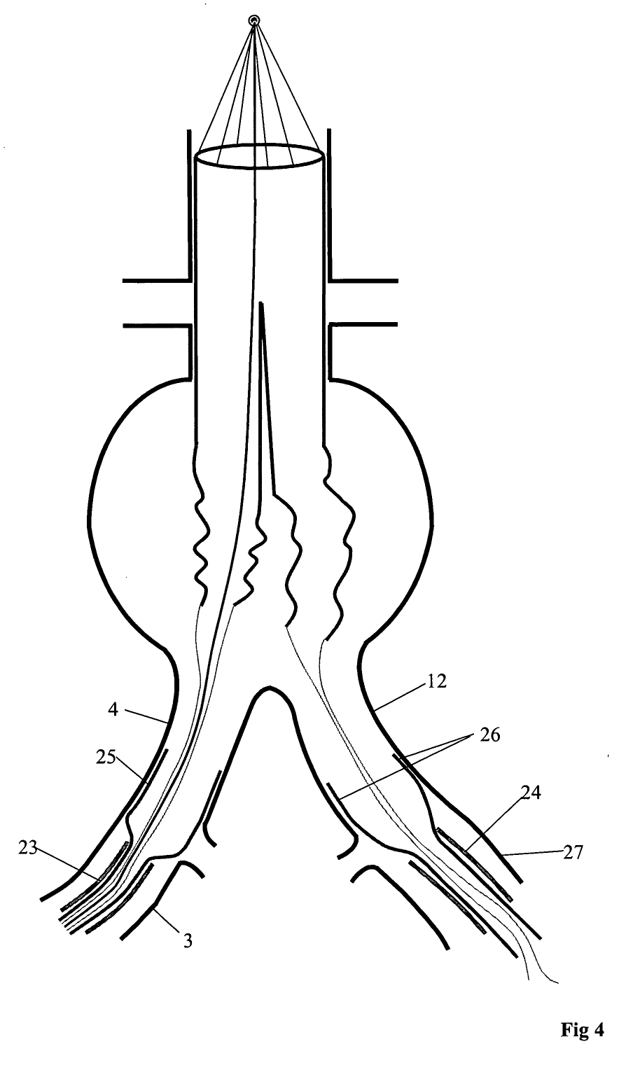

FIG. 4 shows insertion sheaths 23 and 24 withdrawn into external iliac arteries 3 and 27. By withdrawing the restraining effect of insertion sheaths 23 and 24, flared sheaths 25 and 26 have flared in common iliac arteries 4 and 12 respectively.

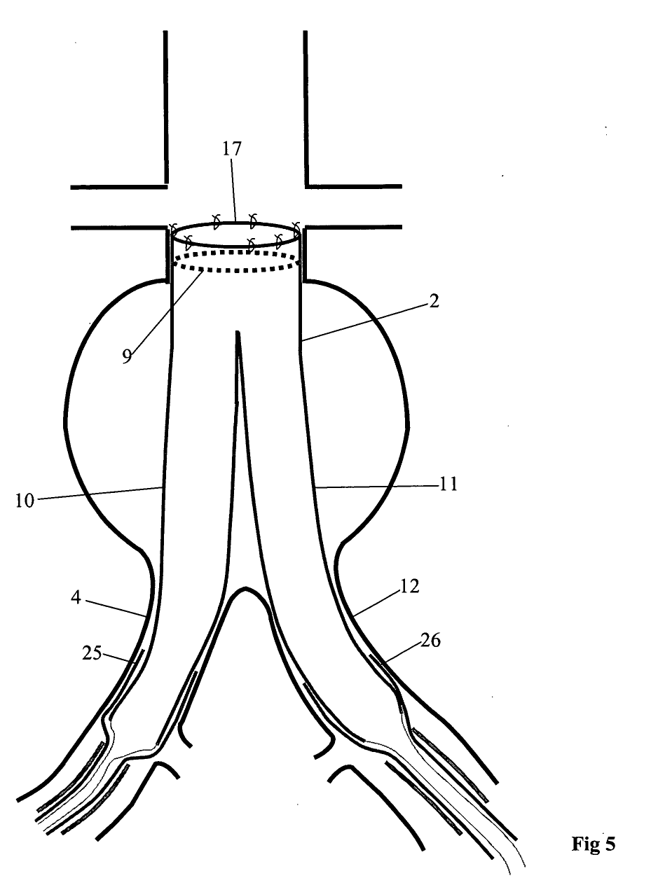

FIG. 5 shows top 17 of endograft 2 attached to aorta 6 neck wall with surgical fasteners 9. Endograft 2 may also be attached to aorta 6 by sutures, fasteners, staples, hooks, or any other suitable attachment method. Endograft limbs 10 and 11 may be too long for the patient and require trimming. The long endograft limbs 10 and 11 have been pulled into flared sheaths 25 and 26 and positioned in right 4 and left 12 common iliac arteries. Endograft limbs 10 and 11 may be pulled into flared sheaths 25 and 26 by use of sutures 21 and 22.

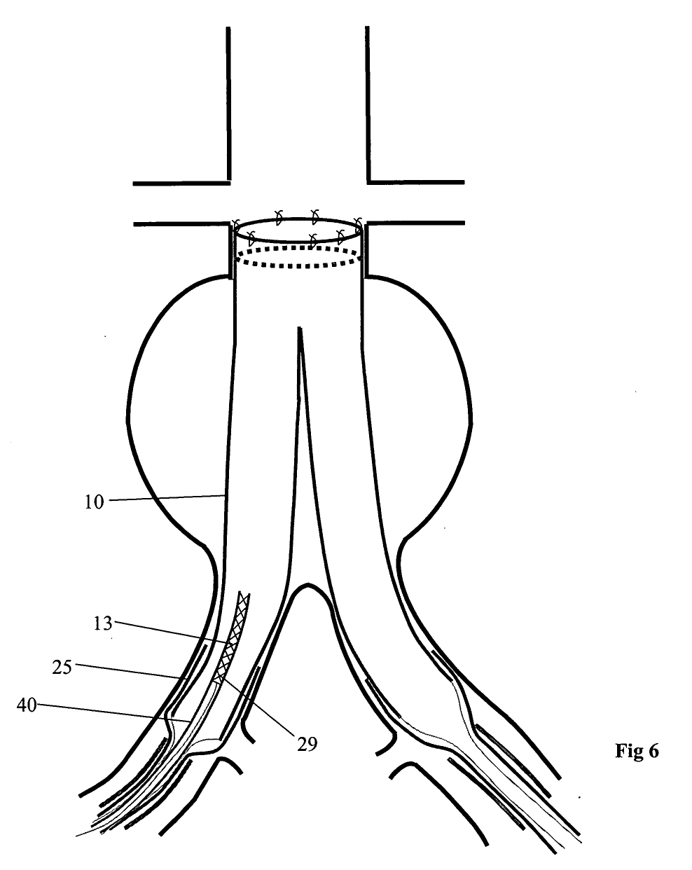

FIG. 6 shows catheter 40 inserted through right flared sheath 25. Catheter 40 comprises a first end, a second end, an inner lumen, and an outer surface. In one embodiment distal end 29 of stent 13 is positioned at the point where it is desired to trim and/or transect endograft limb 10.

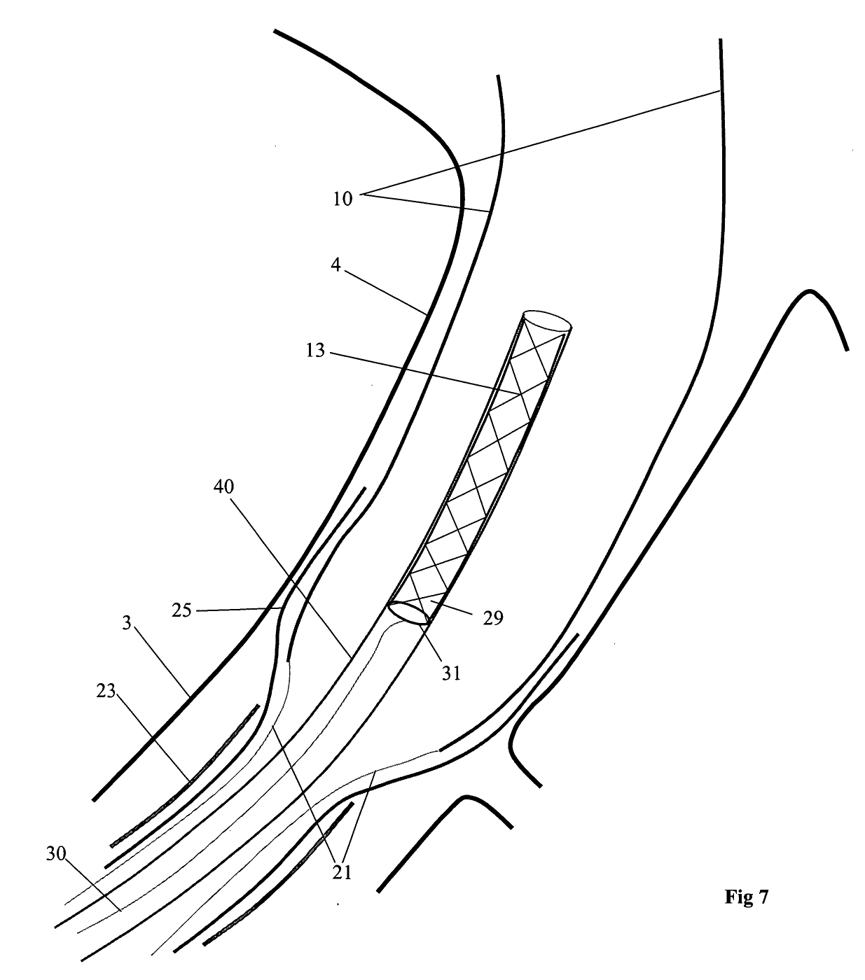

FIG. 7 is a magnified view of right common 4 and external 3 iliac arteries with their contents: right limb 10 of endograft 2 with attached sutures 21, insertion sheath 23, flared sheath 25 inserted through insertion sheath 23, and catheter 40 containing stent 13 within the first end of catheter 40. Attached to distal end 29 of stent 13 is insulated wire 30 leading to filament 31 housed around the circumference of distal end 29 of stent 13. Filament 31 may be comprised of, but not limited to, materials such as tungsten, chromium steel, or any other suitable material. Right common 4 and external 3 iliac arteries, endografts, and components will be used for illustrative purposes in the following figures. The same components, apparatus, and methods may or may not be employed in the left common 12 and external 27 iliac arteries.

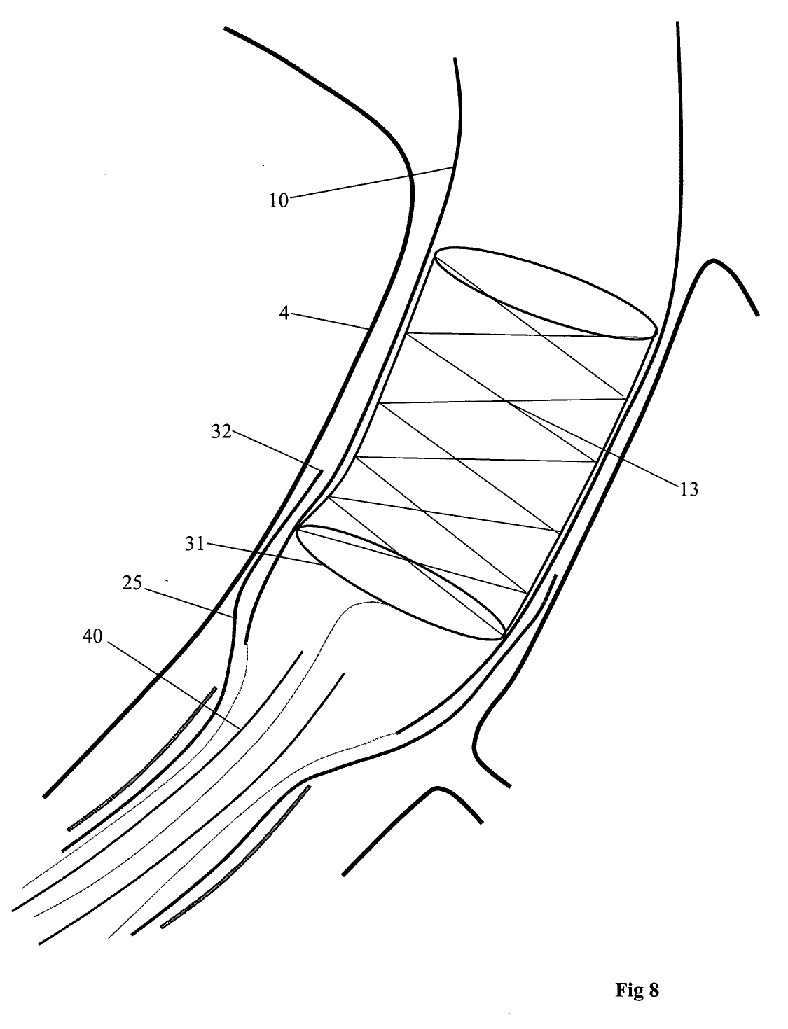

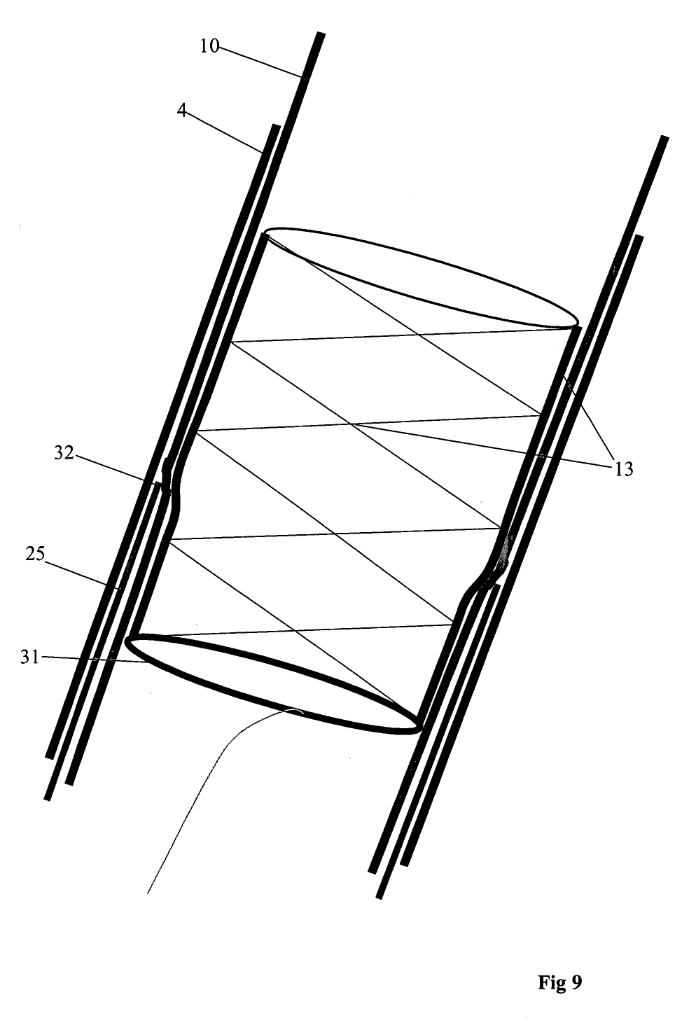

FIG. 8 shows stent 13 unsheathed from catheter 40 and expanded such that it compresses endograft limb 10 to common iliac artery 4 from the proximal end of stent 13 ( proximal to the heart) to first end 32 (distal to the interventionalist, proximal to the heart) of flared sheath 25. It also compresses endograft limb 10 to the portion of the flared sheath 25 from first end 32 to the end of distal end 29 of stent 13. These relationships are also diagramed in FIG. 9 to further demonstrate the relationships when the various layers are drawn immediately adjacent to one another as they would be when stent 13 is in its sufficiently dilated configuration.

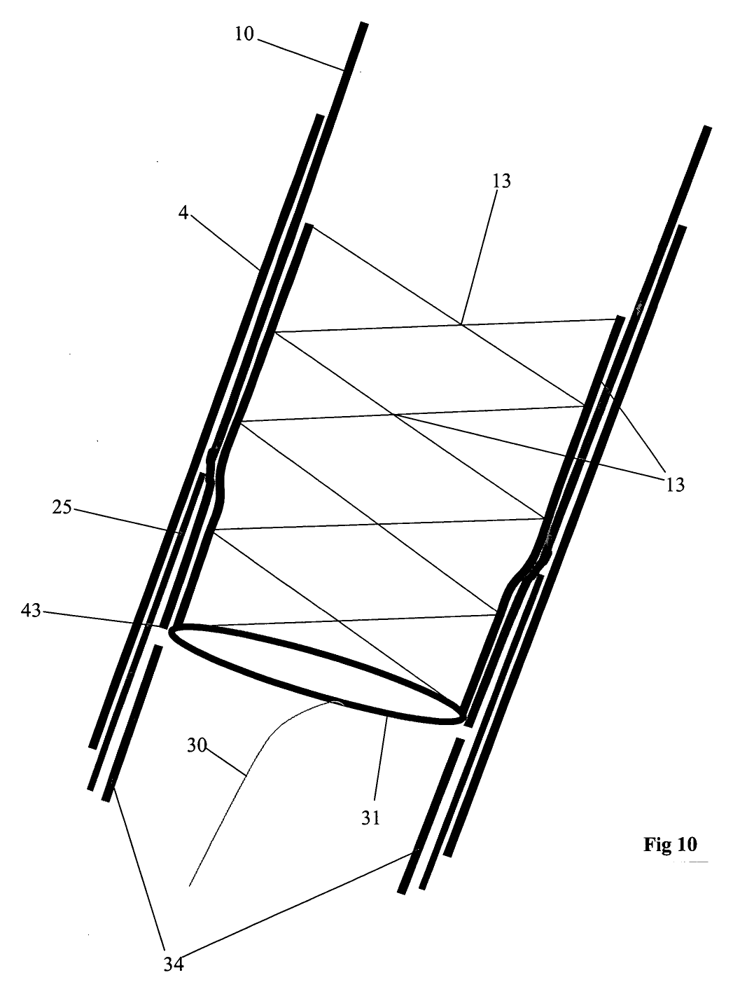

FIG. 10 depicts a trimming or transection of endograft limb 10 at position 43 thus detaching excess endograft material 34 of endograft limb 10 distal to distal end 29 of stent 13. In an embodiment of the present invention this is achieved by applying a current to insulated wire 30 that is attached to filament 31 imbedded on the outside portion of distal end 29 of stent 13. This will heat filament 31 sufficiently to burn through the material of endograft 2 that is in immediate contact with filament 31 disposed in stent 13. Flared sheath 25 serves to protect common iliac artery wall 4 at the level of heated filament 31 disposed in distal end 29 of stent 13.

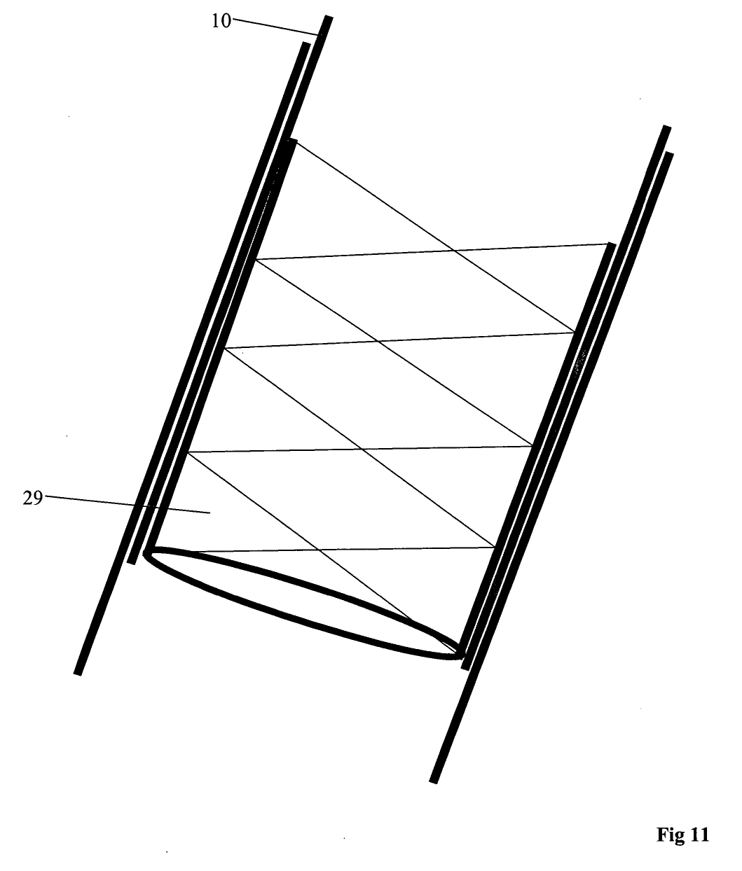

FIG. 11 shows the shortened endograft limb 10 cut at the distal end 29 of stent 13. Excess endograft material 34, insulated wire 30 and flared sheath 25 have been removed. Insulated wire 30 may be detached from filament 31 by any suitable means, including, but not limited to, cutting, or filament 31 may be withdrawn from stent 13 along with insulated wire 30.

In an embodiment of the present invention, stent 13 could be equipped with alternative means of transecting endograft limb 10. In addition to heat, endograft limb 10 could be transected using any mechanical, electrical, or optical force, including but not limited to, lasers, mechanical cutting, or any other suitable method that can be adapted for use in stent 13.

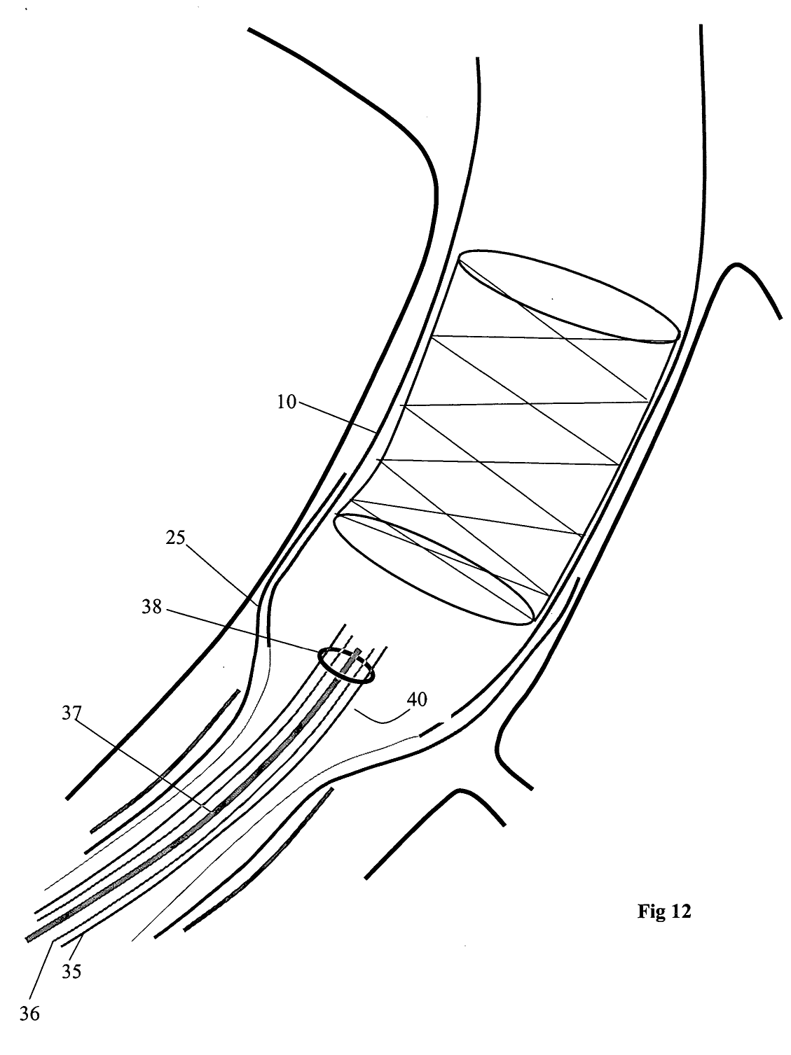

FIGS. 12-14 show one embodiment of the apparatus and method of transecting a endograft limb. In FIG. 12, catheter 40 comprising outer sheath 35, inner sheath 36 and optical fiber 37 is inserted through flared sheath 25. Inner sheath 36 and outer sheath 35 both comprise a first end, a second end, an inner lumen, and an outer surface. Optical fiber 37 comprises a first end and a second end. Expandable housing 38 may be disposed on a proximal portion, or first end, of outer sheath 35. Expandable housing 38 may comprise a balloon, expandable and retractable struts, or any other similar expandable or stabilizing mechanism.

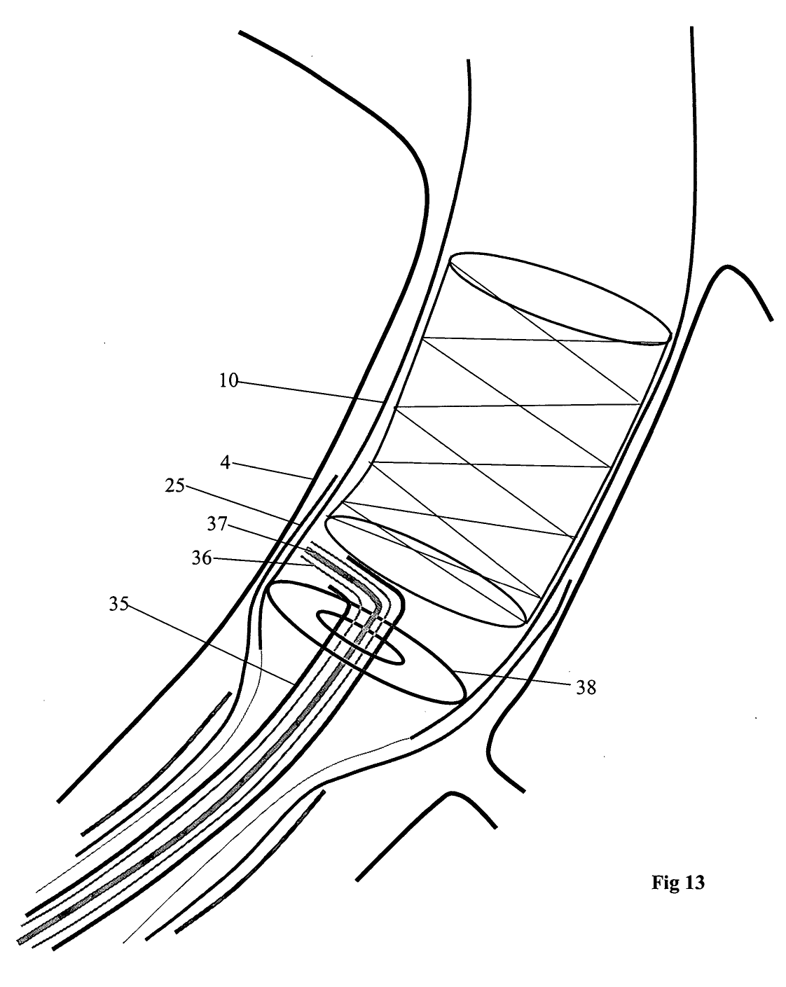

FIG. 13 shows housing 38 expanded to compress endograft limb 10 against flared sheath 25, which, in turn, is compressed against the inner portion of common iliac artery 4 wall. Outer sheath 35 is tip deflected and inner sheath 36 and optical fiber 37 are advanced within outer sheath 35 until they are close to or touching endograft limb 10.

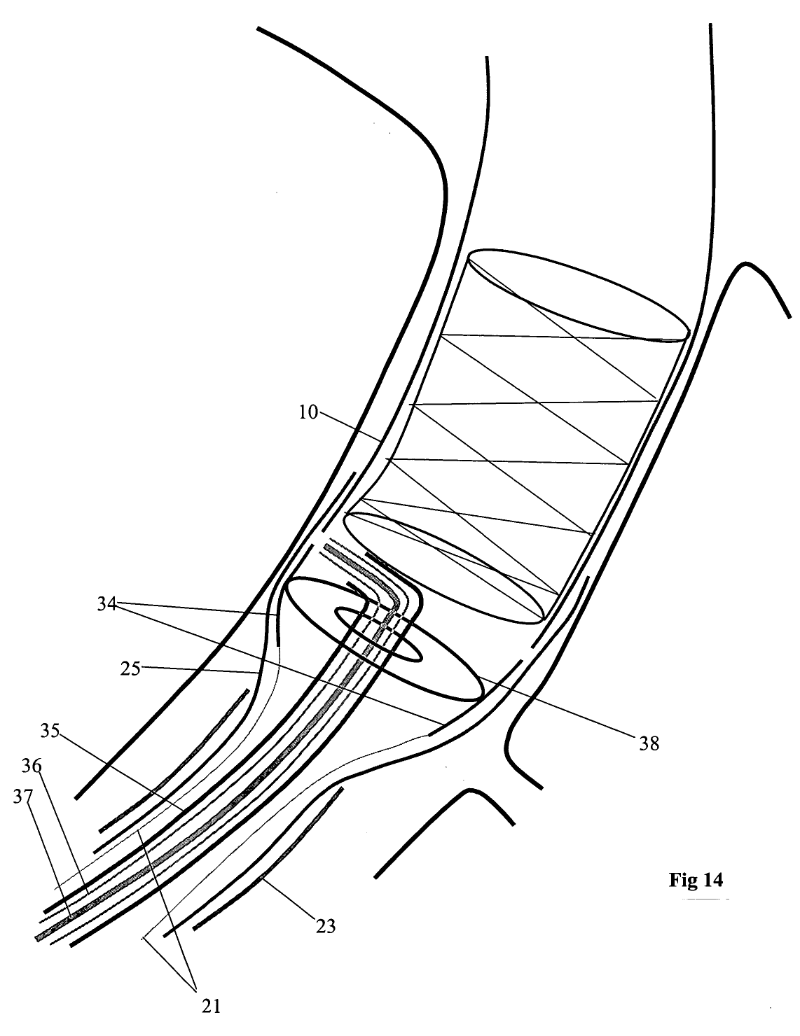

The laser and optical fiber 37 are activated and outer sheath 35 is rotated circumferentially until endograft limb 10 is transected as depicted in FIG. 14. After endograft limb 10 is transected, outer sheath 35 is straightened, housing 38 is retracted and catheter 40 components 35, 36, 37, flared sheath 25, insertion sheath 23 and transected excess material 34 of endograft limb 10 and attached sutures 21 are removed.

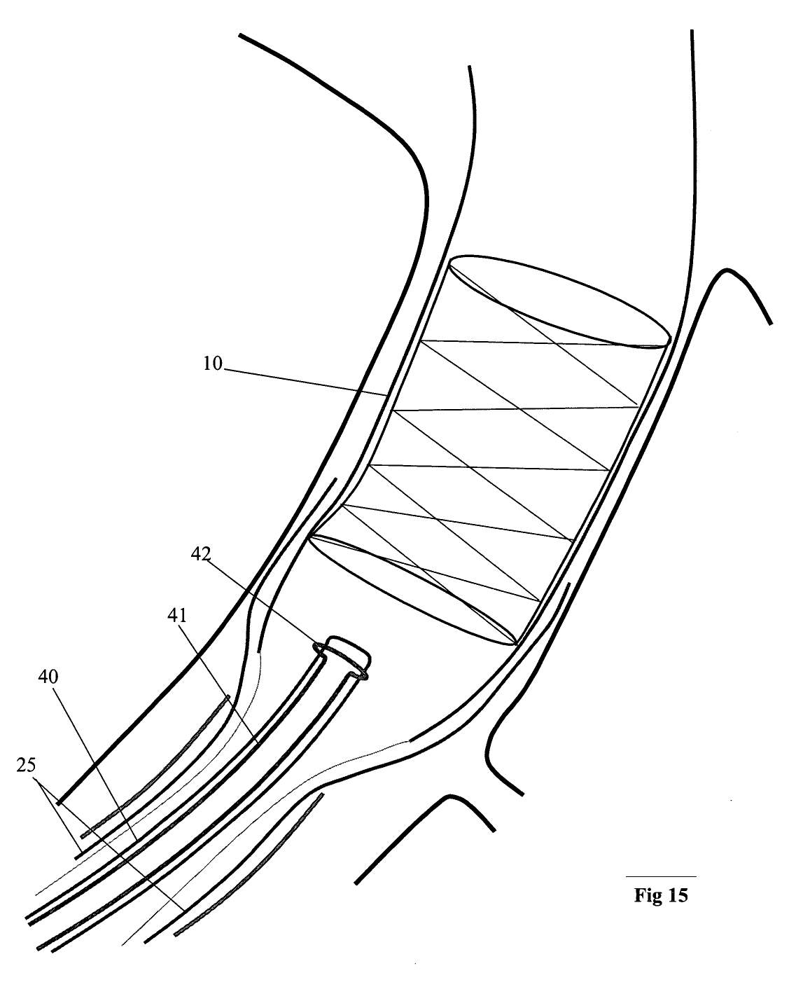

An embodiment for transecting endograft limb 10 is depicted in FIGS. 15-17. In FIG. 15 catheter 40 comprises wire 41 with a first end, a second end, and a mid portion disposed between the first end and the second end. The mid portion of wire 41 extends, for example, from at least one opening 44 disposed in catheter 40 near its leading edge, or first end, and extends around the outer surface of catheter 40. The first end of catheter 40 comprising wire 41 is inserted through flared sheath 25. Wire 41 may comprise an insulated portion on all or part of wire 41 within catheter 40, or only on one surface of wire 41. Insulating material may comprise, but is not limited to, polyurethane, and/or any other suitable insulating material. Wire 41 may incorporate filament 31 on the portion of wire 41 that will be extended from catheter 40 during the surgical procedure. Filament 31 may be insulated such that the outer circumference of filament 31 insulated, or such that the inner circumference of filament 31 is insulated. Catheter 40 also may have an inner lumen so it can be passed over a guidewire (not shown). The first end of catheter 40 is inserted through flared sheath 25 and advanced into position within endograft limb 10.

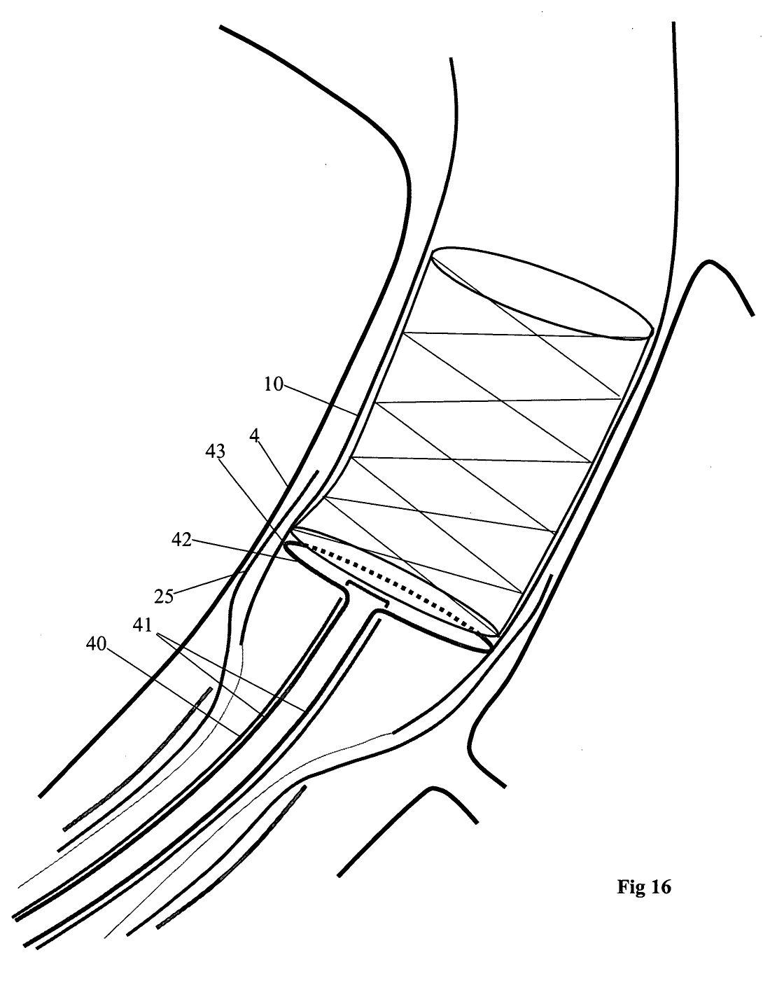

FIG. 16 depicts wire 41 having been advanced through catheter 40 such that wire ring 42 is advanced to compress endograft limb 10 at a position, for example, position 43 of desired transection. Ring 42 comprises filament 31 on its outer surface, or outer circumference, such that, when a current is passed through wire 41 and filament 31, filament 31 will heat sufficiently to transect endograft limb 10 by contact burning. Filament 31 may be insulated on its inner surface, or inner circumference. Flared sheath 25 is in position to prevent any burn damage to the adjacent common iliac artery 4 wall. The entire length of wire 41 may comprise filament 31, or only portions of wire 41 that will be exposed to transect endograft 2 may comprise filament 31. In an alternative embodiment of the present invention, ring 42 is disposed between flared sheath 25 and endograft limb 10. In this embodiment limb 10 is transected by the action of filament 31 on the outer surface of limb 10 as ring 42 is drawn into contact with limb 10 by reducing the diameter of ring 42. In this embodiment, the outer surface or outer circumference of filament 31 may be insulated, with the inner surface or inner circumference capable of cutting limb 10.

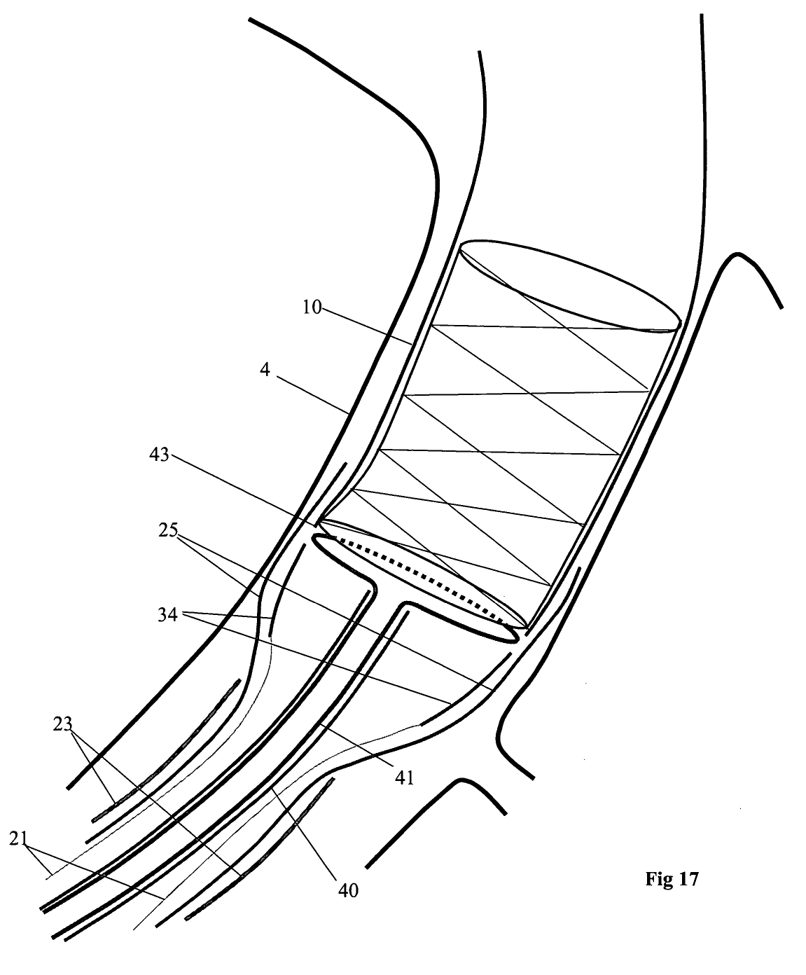

FIG. 17 shows endograft limb 10 transected at position 43 near distal end 29 of stent 13. After endograft limb 10 is transected, wire 41 comprising filament 31 are retracted into catheter 40. Catheter 40, flared sheath 25, insertion sheath 23 and transected excess material 34 portion of endograft 2 and attached sutures 21 are removed.

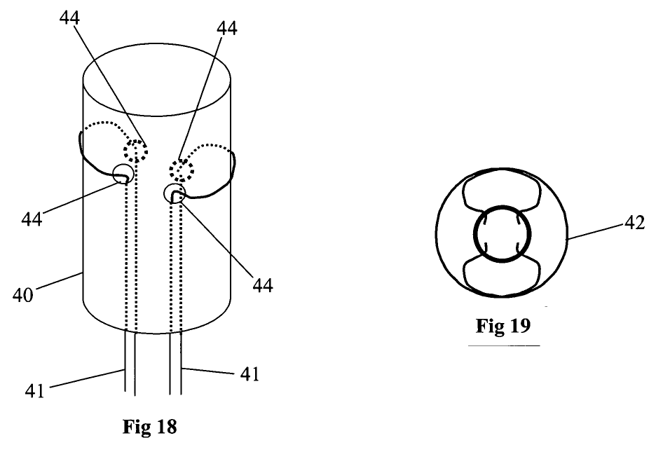

In an embodiment it may be advantageous to have at least two sets of wire 41 comprising filament 31 disposed within catheter 40. FIG. 18 depicts catheter 40 with two sets of wire 41 comprising filament 31 and four exit ports 44 near the leading edge, or first end, of catheter 40. As shown, a first pair of ports 44 are slightly nearer the first end of catheter 40 than a second pair of ports 44. When wires 41 comprising filament 31 are advanced through catheter 40, two rings 42 are formed as shown in FIG. 19. FIG. 19 shows rings 42 as they would appear from above when both sets of wires 41 are advanced at the same time thus forming a circle which, when looked at laterally, would show a first set of wires 41 slightly above a second set of wires 41. Filament 31 is exposed along the portion of wires 41 that contact the graft material to be cut.

Alternatively, in an embodiment of the present invention, the catheter could be equipped with alternative means of transecting the endograft limb. In addition to heat and lasers, the endograft could be transected using any mechanical, electrical, or optical force, including but not limited to mechanical cutting, or any other suitable method that can be adapted for the catheter.

Numerous characteristics and advantages have been set forth in the foregoing description, together with details of structure and function. The novel features are pointed out in the appended claims. The disclosure, however, is illustrative only, and changes, may be made in detail, especially in matters of shape, size, and arrangement of parts, within the principle of the invention, to the full extent indicated by the broad general meaning of the terms in which the appended claims are expressed.

What is claimed is:

1. An apparatus for endovascularly cutting a graft comprising:
a catheter having a first end, a second end, an inner lumen, and an outer surface, further comprising at least one opening near its first end;
at least one wire further comprising a filament extending through the at least one opening and around an outer surface of the catheter;
wherein the wire is movable within the catheter and can be extended to form a ring disposed a predetermined distance around the outer surface of the catheter.

2. The apparatus of claim 1 wherein the wire has a first end, a second end, and a mid portion disposed between the first and second ends, wherein the mid portion extends from the at least one opening.

3. The apparatus of claim 1 further comprising a flared sheath wherein the first end of the catheter is inserted into the flared sheath.

4. The apparatus of claim 1 wherein the wire further comprises a filament.

5. The apparatus of claim 4 wherein the filament is tungsten.

6. An apparatus for endovascularly cutting a graft comprising:
a stent having a distal end;
a catheter having a first end, a second end, an inner lumen, and an outer surface;
a filament disposed within the circumference of the distal end of the stent;
a wire having a first end and a second end, wherein the first end is in communication with the filament and the second end extends away from the stent into the lumen of the catheter.

7. The apparatus of claim 6 further comprising a flared sheath wherein the first end of the catheter is inserted into the flared sheath.

8. The apparatus of claim 6 wherein the wire and the filament are detachable.

9. The apparatus of claim 6 wherein the filament is tungsten.

10. The apparatus of claim 6 wherein a portion of the wire is insulated.

11. An apparatus for endovascularly cutting a graft comprising:
a flared sheath having a first flared end, a second end, an inner lumen, and an outer surface;
a catheter having a first end, a second end, an inner lumen, and an outer surface, wherein a portion of the catheter is disposed within the inner lumen of the flared sheath;
an inner sheath having a first end, a second end, an inner lumen, and an outer surface, wherein a portion of the inner sheath is disposed within the inner lumen of the catheter; and
an optical fiber having a first end and a second end, wherein a portion of the optical fiber is disposed within the inner sheath.

12. The apparatus of claim 11 wherein the flared sheath is composed of a metal.

13. The apparatus of claim 11 wherein the flared sheath is composed of a polyamide.

14. The apparatus of claim 11 further comprising an outer sheath.

15. The apparatus of claim 14 further comprising an expandable housing disposed on the outer sheath.

16. A method for endovascularly cutting a graft comprising the steps of:
inserting a catheter having a first end, a second end, an inner lumen, and an outer surface, further comprising at least one opening near its first end and at least one wire further comprising a filament extending through the at least one opening and around an outer surface of the catheter;
extending the wire comprising the filament to form a ring disposed a predetermined distance around the outer surface of the catheter and contacting the filament to a portion of the graft to be cut; and
applying a current to the wire and the filament such that the filament cuts the graft.

17. The method of claim 16 wherein a portion of the wire and the filament are insulated.

18. The method of claim 16 wherein the catheter has at least two openings.

19. The method of claim 16 wherein the catheter has at least two wires.

20. The method of claim 16 wherein the filament cuts the graft from the inner surface of the graft.
